(19) United States
(12) Patent Application Publication
(10) Pub. No.: US 2024/0331849 A1
(43) Pub. Date: Oct. 3, 2024

(54) COMPUTER-READABLE RECORDING MEDIUM STORING MEDICAL IMAGE PROCESSING PROGRAM, MEDICAL IMAGE PROCESSING METHOD, AND INFORMATION PROCESSING DEVICE

(71) Applicant: Fujitsu Limited, Kawasaki-shi (JP)

(72) Inventors: Jumpei YAMANAKA, Adachi (JP); Masaki TAKEUCHI, Kawasaki (JP); Yuhi OTANI, Kawasaki (JP); John MAGNUSSEN, North Ryde (AU)

(73) Assignee: Fujitsu Limited, Kawasaki-shi (JP)

(21) Appl. No.: 18/740,149

(22) Filed: Jun. 11, 2024

Related U.S. Application Data
(63) Continuation of application No. PCT/JP2021/048392, filed on Dec. 24, 2021.

(51) Int. Cl. G16H 30/40; G16H 50/20

(52) U.S. Cl. CPC G16H 30/40; G16H 50/20

(57) ABSTRACT

A non-transitory computer-readable recording medium stores a medical image processing program for causing a computer to execute a process including: narrowing down lesion candidate regions detected by a detection model that detects the lesion candidate regions included in a medical image, by using a first threshold value on certainty factors calculated by the detection model; and determining false positives of the lesion candidate regions by comparing the certainty factors calculated for the lesion candidate regions with a second threshold value subdivided according to an index other than the certainty factors.

### CROSS-REFERENCE TO RELATED APPLICATION

This application is a continuation application of International Application PCT/JP2021/048392 filed on Dec. 24, 2021 and designated the U.S., the entire contents of which are incorporated herein by reference.

### FIELD

The present embodiment relates to a medical image processing program, a medical image processing method, and an information processing device.

### BACKGROUND

In recent years, a computer aided detection (CADe) technology for detecting an anomalous location, using a computer, in a medical image such as a computed tomography (CT) image has been used. In the CADe, many attempts have been made to detect an anomalous region by processing a medical image using deep learning (DL).

Related art is disclosed in Japanese Laid-open Patent Publication No. 2011-104206, International Publication Pamphlet No. WO 2007/026598, Japanese Laid-open Patent Publication No. 2020-171480, Japanese Patent No. 3928978 and Yang, Jiehua, et al., “Deep learning for detecting cerebral aneurysms with CT angiography.”, Radiology, 298.1 (2021): 155-163.

### SUMMARY

According to an aspect of the embodiments, a non-transitory computer-readable recording medium stores a medical image processing program for causing a computer to execute a process including: narrowing down lesion candidate regions detected by a detection model that detects the lesion candidate regions included in a medical image, by using a first threshold value on certainty factors calculated by the detection model; and determining false positives of the lesion candidate regions by comparing the certainty factors calculated for the lesion candidate regions with a second threshold value subdivided according to an index other than the certainty factors.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram depicting a hardware configuration of a medical image processing system as an example of an embodiment.

FIG. 2 is a diagram depicting a functional configuration of the medical image processing system as an example of the embodiment.

FIG. 3 is a diagram schematically illustrating a process of a machine learning model of the medical image processing system as an example of the embodiment.

FIG. 4 is a diagram for explaining a first narrowing-down process in the medical image processing system as an example of the embodiment.

FIG. 5 is a flowchart for explaining a process in the medical image processing system as an example of the embodiment.

### DESCRIPTION OF EMBODIMENTS

For example, pixel spacing normalization and cropping are performed as preprocessing on a medical image, and an anomaly candidate is detected as a bounding box, using a three-dimensional (3D) convolutional neural network (CNN) shadow detector. Thereafter, overlapping candidates are reduced in post-processing, which is known as an approach. This aids in the detection of cerebral aneurysms in CT angiography (CTA) images.

In addition, based on the knowledge that a cerebral aneurysm and a false positive are likely to occur at a certain position, there is known an approach of reducing false positives from regions of detected cerebral aneurysm candidates, using a feature indicating position coordinates, to thereby reduce false positive candidates in detection of a cerebral aneurysm.

Furthermore, as a method of aiding in detecting a cerebral aneurysm candidate from a magnetic resonance (MR) blood vessel image, there is also known an approach of performing false positive removal on a candidate point for a cerebral aneurysm identified from a luminance change (vector) between a blood vessel and a voxel outside the blood vessel, according to a difference between an internal region and an external region relating to a feature (deviation, mean, maximum, or minimum) of luminance.

However, in such conventional CADe technologies, for example, in estimation of an aneurysm on a CT image, for a lesion position estimated by a stochastic statistical approach such as a CNN, there is a case where a portion that is obviously not the aneurysm, such as the vein, is erroneously estimated (false positive) according to an imaging condition such as the administration timing of a contrast medium.

For example, under an appropriate condition, the vein is supposed to be less likely to appear because imaging is performed before the contrast medium flows into the vein, but if the administration timing of the contrast medium is shifted, the vein is also imaged brightly to some extent in contrast imaging.

In addition, for example, based on the knowledge that a cerebral aneurysm and a false positive are likely to occur at a certain position, there is known an approach of reducing false positives from regions of detected cerebral aneurysm candidates, using a feature indicating position coordinates. When utilizing such knowledge for false positive reduction, positional alignment is involved. However, for example, there are a place where the cerebral aneurysm is likely to be formed and a place where the cerebral aneurysm is less likely to be formed. For this reason, positional alignment is involved, but processing sometimes may not be successful depending on the accuracy of positional alignment.

In addition, in an approach of performing false positive removal on a candidate point for a cerebral aneurysm according to a difference between an internal region and an external region relating to a feature of luminance, the candidate point is deduced after blood vessel extraction. For this reason, a modality that is easy to clearly extract a blood vessel, such as MR angiography (MRA), is targeted, and it is difficult to apply such a modality to a case where a portion other than the blood vessel is likely to appear, like the CTA.

In one aspect, an object of the present invention is to enable determination of a false positive of a lesion candidate region detected in a medical image, with high accuracy.

Hereinafter, embodiments according to the present medical image processing program, medical image processing method, and information processing device will be described with reference to the drawings. However, the embodiments to be described below are merely examples, and there is no intention to exclude application of various modifications and techniques not explicitly described in the embodiments. That is, the present embodiments can be variously modified and carried out in a range without departing from the spirit of the embodiments. In addition, each drawing is not intended to include only components illustrated in the drawing and can include other functions and the like.

### (A) Configuration

A medical image processing system 1 as an embodiment of the present invention implements a function of determining a false positive of a lesion focus candidate estimated in a medical image.

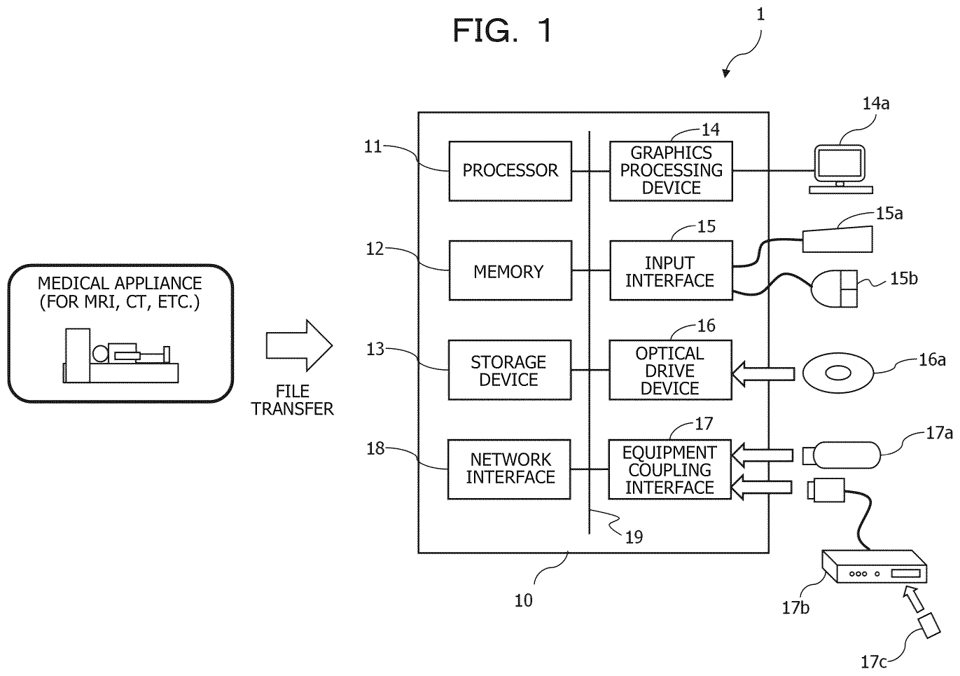

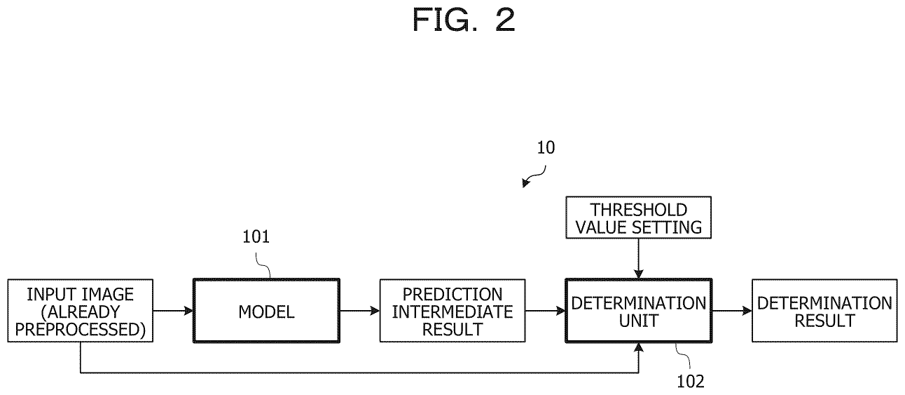

FIG. 1 is a diagram depicting a hardware configuration of the medical image processing system 1 as an example of the embodiment, and FIG. 2 is a diagram depicting a functional configuration of the medical image processing system 1.

As illustrated in FIG. 1, the present medical image processing system 1 includes an information processing device 10. A medical image captured by a medical appliance for magnetic resonance imaging (MRI), CT, or the like is input to the information processing device 10.

In the following, an example in which the medical image is a three-dimensional CTA medical image of the head of a patient and lesion of a cerebrovascular disease including a cerebral aneurysm is detected will be described.

The information processing device 10 is a computer and includes, for example, a processor 11, a memory 12, a storage device 13, a graphics processing device 14, an input interface 15, an optical drive device 16, an equipment coupling interface 17, and a network interface 18 as components, as illustrated in FIG. 1. These components 11 to 18 are configured so as to be able to mutually communicate via a bus 19.

The processor (control unit) 11 controls the entire information processing device 10. The processor 11 may be a multiprocessor. The processor 11 may be, for example, any one of a central processing unit (CPU), a micro processing unit (MPU), a digital signal processor (DSP), an application specific integrated circuit (ASIC), a programmable logic device (PLD), and a field programmable gate array (FPGA). In addition, the processor 11 may be a combination of two or more types of elements of the CPU, MPU, DSP, ASIC, PLD, FPGA, and a graphics processing unit (GPU).

Then, the processor 11 executes a program (a medical image processing program, an OS program) recorded in, for example, a computer-readable non-transitory recording medium, whereby the functions as a machine learning model (model) 101 and a determination unit 102 depicted in FIG. 2 are implemented. The OS is an abbreviation for an operating system.

The program in which processing contents to be executed by the information processing device 10 are described can be recorded in a variety of recording media. For example, the program to be executed by the information processing device 10 can be stored in the storage device 13. The processor 11 loads at least a part of the program in the storage device 13 into the memory 12 and executes the loaded program.

In addition, the program to be executed by the information processing device 10 (processor 11) can also be recorded in a non-transitory portable recording medium such as an optical disc 16a, a memory device 17a, or a memory card 17c. The program stored in the portable recording medium becomes executable after being installed in the storage device 13 under the control of the processor 11, for example. In addition, the processor 11 can also directly read and execute the program from the portable recording medium.

The memory 12 is a storage memory including a read only memory (ROM) and a random access memory (RAM). The RAM of the memory 12 is used as a main storage device of the information processing device 10. The RAM temporarily stores at least a part of the program to be executed by the processor 11. In addition, the memory 12 stores various types of data involved in processing by the processor 11.

The storage device 13 is a storage device such as a hard disk drive (HDD), a solid state drive (SSD), or a storage class memory (SCM) and stores various kinds of data. The storage device 13 is used as an auxiliary storage device of the information processing device 10.

The storage device 13 stores the OS program, a control program, and various types of data. The control program includes the medical image processing program.

Note that a semiconductor storage device such as an SCM or a flash memory can also be used as an auxiliary storage device. In addition, redundant arrays of inexpensive disks (RAID) may be configured using a plurality of the storage devices 13.

A monitor 14a is coupled to the graphics processing device 14. The graphics processing device 14 displays an image on a screen of the monitor 14a in accordance with a command from the processor 11. Examples of the monitor 14a include a display device using a cathode ray tube (CRT), a liquid crystal display device, and the like.

A keyboard 15a and a mouse 15b are coupled to the input interface 15. The input interface 15 transmits signals sent from the keyboard 15a and the mouse 15b to the processor 11. Note that the mouse 15b is an exemplary pointing device, and another pointing device can also be used. Examples of the another pointing device include a touch panel, a tablet, a touch pad, a track ball, and the like.

The optical drive device 16 reads data recorded in the optical disc 16a, using laser light or the like. The optical disc 16a is a non-transitory portable recording medium in which data is recorded in a readable manner by reflection of light. Examples of the optical disc 16a include a digital versatile disc (DVD), a DVD-RAM, a compact disc read only memory (CD-ROM), a CD-recordable (R)/rewritable (RW), and the like.

The equipment coupling interface 17 is a communication interface for coupling peripheral equipment to the information processing device 10. For example, the memory device 17a and a memory reader/writer 17b can be coupled to the equipment coupling interface 17. The memory device 17a is a non-transitory recording medium equipped with a function of communicating with the equipment coupling interface 17, such as a universal serial bus (USB) memory. The memory reader/writer 17b writes data to the memory card 17c or reads data from the memory card 17c. The memory card 17c is a card-type non-transitory recording medium.

The network interface 18 is coupled to a network. The network interface 18 transmits and receives data via the network. Other information processing devices, communication equipment, or the like may be coupled to the network. For example, the information processing device 10 may be coupled to a medical appliance via the network interface 18 and the network and receive a medical image from this medical appliance by file transfer.

In addition, the information processing device 10 may be coupled to a database system that stores medical images via the network interface 18 and the network and receive the medical images from this database system.

A medical image that has undergone preprocessing (already preprocessed) by a preprocessing unit (not illustrated) is input to the machine learning model 101.

The preprocessing for the medical image may include rescaling for altering the size of the medical image to a size for treatment, for example. In the rescaling, the distance between pixels may be adjusted in all directions in the medical image.

In addition, the preprocessing for the medical image may include cropping or padding. For example, when the size of the input medical image is larger than a predetermined size (for example, 256×256×256), a central portion may be cropped. Meanwhile, when the size of the input medical image is equal to or smaller than the predetermined size, the imaging target may be padded so as to be located at the center. Furthermore, the preprocessing for the medical image may include a gradation process (windowing) and normalization of contrast.

Hereinafter, unless otherwise explicitly specified, the medical image will refer to a medical image that has been already preprocessed. The preprocessing unit may be provided outside the present medical image processing system 1 or alternatively, may be provided as one function of the present medical image processing system 1.

The machine learning model 101 is an estimation model that is implemented by using a neural network and estimates an aneurysm candidate in a medical image with a convolutional neural network (CNN). In addition, the machine learning model 101 can also be referred to as a detection model that detects an aneurysm candidate included in a medical image. When a medical image is input, the machine learning model 101 outputs a bounding box indicating a position estimated as an affected part (affected part candidate) of an aneurysm in the input medical image. The bounding box is a lesion candidate region relevant to the disease to be detected. The bounding box may be referred to as an affected part candidate. In addition, the bounding box may be represented as a BB or, alternatively, may be represented as an aneurysm candidate BB. The machine learning model 101 also outputs each of luminance values of a plurality of pixels included in the bounding box.

The machine learning model 101 estimates an aneurysm candidate on the input CTA medical image and outputs the estimated aneurysm candidate as a bounding box.

Note that the creation of the bounding box indicating the affected part candidate can be implemented by a known approach such as Yang, Jiehua, et al., “Deep learning for detecting cerebral aneurysms with CT angiography.”, Radiology, 298.1 (2021): 155-163 (Non-Cited Document 1), for example, and description thereof will be omitted.

In addition, in the present embodiment, an example in which the bounding box is a cube will be described.

Information on a bounding box indicating a position estimated as an affected part (affected part candidate) of an aneurysm in the medical image and each of the luminance values of the plurality of pixels included in the bounding box, which are output from the machine learning model 101, may be referred to as a prediction intermediate result.

The prediction intermediate result output from the machine learning model 101 is stored in a predetermined storage area of the memory 12, the storage device 13, or the like.

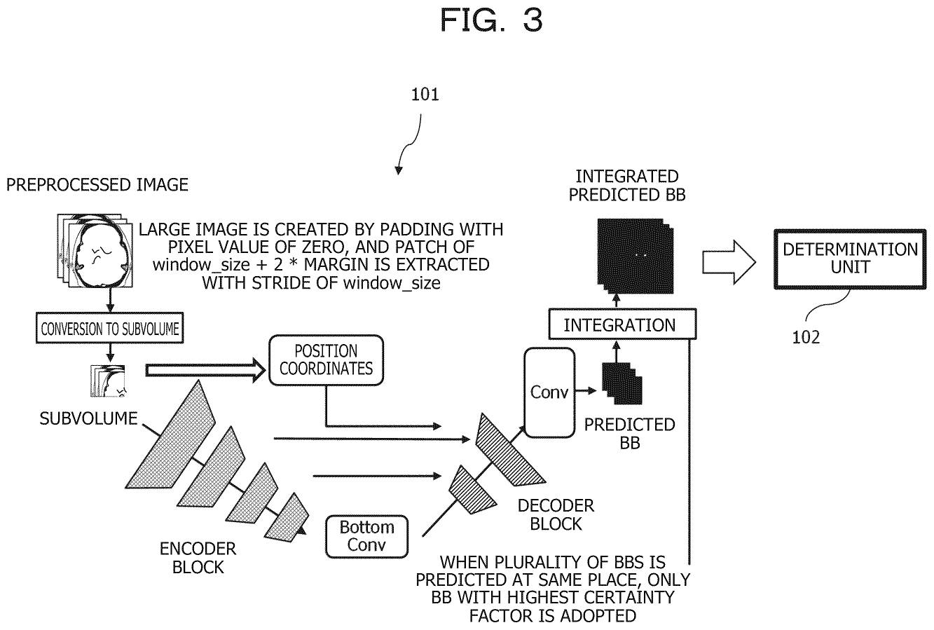

FIG. 3 is a diagram schematically illustrating a process of the machine learning model 101 of the medical image processing system 1 as an example of the embodiment.

In this example illustrated in FIG. 3, the machine learning model 101 includes a 3D convolutional neural network shadow detector and detects an anomaly candidate as a bounding box.

The machine learning model 101 converts the preprocessed medical image into a subvolume and extracts a feature with an encoder. In addition, a decoder restores the extracted feature. Predicted bounding boxes are integrated for each subvolume.

Note that, in a case where a plurality of bounding boxes is predicted at the same place, a bounding box with the highest certainty factor is adopted.

The prediction intermediate result output from the machine learning model 101 is input to the determination unit 102. The determination unit 102 determines whether an affected part candidate included in each bounding box is true positive or false positive, for each bounding box (lesion candidate region) output from the machine learning model 101.

The determination unit 102 narrows down aneurysm candidates each estimated by the machine learning model 101 and indicated as a bounding box, using the certainty factors and statistical values of luminance values (Hounsfield unit values: HU values) of a plurality of pixels included in the bounding box. In the present embodiment, an example of using, as the statistical value, a mean value of the luminance values of a plurality of pixels included in the bounding box will be described.

The determination unit 102 narrows down a plurality of bounding boxes output from the machine learning model 101 to a bounding box that satisfies a first condition that a certainty factor c is higher than a first certainty factor threshold value T_{C1} and a mean value I_{μ} of the luminance values of the input pixels in the bounding box is higher than a first luminance threshold value T_{I1}. The first certainty factor threshold value T_{C1} corresponds to a first threshold value. In addition, the first luminance threshold value T_{I1} corresponds to a third threshold value.

That is, in a case where a bounding box output from the machine learning model 101 does not satisfy the first condition that the certainty factor c is higher than the first certainty factor threshold value T_{C1} and the mean value I_{μ} of the luminance values of the input pixels in the bounding box is higher than the first luminance threshold value T_{I1}, the determination unit 102 excludes the bounding box (affected part candidate) by regarding the bounding box as a false positive. The first certainty factor threshold value T_{C1} may be simply referred to as a certainty factor threshold value T_{C1}.

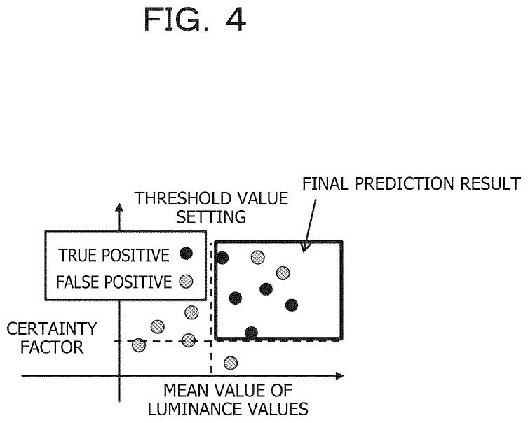

FIG. 4 is a diagram for explaining a first narrowing-down process in the medical image processing system 1 as an example of the embodiment.

In FIG. 4, a two-dimensional coordinate space of which the horizontal axis denotes the mean value of the luminance values of the plurality of pixels included in the bounding box and the vertical axis denotes the certainty factor is illustrated, and a plurality of affected part candidates (bounding boxes) is plotted in this two-dimensional coordinate space. These affected part candidates indicate either a true positive or a false positive.

The present medical image processing system 1 narrows down the aneurysm candidates estimated by the machine learning model 101, based on an empirical rule that affected part candidates having a higher certainty factor include many true positives and additionally, affected part candidates having a higher statistical value of the luminance values of a plurality of pixels included in the bounding box include many true positives.

Specifically, the first luminance threshold value T_{I1} is provided for the luminance value, and an affected part candidate of which the statistical value of the luminance values of a plurality of pixels included in the bounding box is equal to or lower than this first luminance threshold value T_{I1} is regarded as a false positive and excluded. The determination unit 102 selects an affected part candidate of which the statistical value of the luminance values of the plurality of pixels included in the bounding box is higher than the first luminance threshold value TI. The first luminance threshold value T_{I1} may be simply referred to as a luminance threshold value T_{I1}.

In addition, the first certainty factor threshold value T_{C1} is provided for the certainty factor, and an affected part candidate of which the certainty factor is equal to or lower than this first certainty factor threshold value T_{C1} is regarded as a false positive and excluded. The determination unit 102 selects an affected part candidate of which the certainty factor is higher than the first certainty factor threshold value T_{C1}.

In this manner, the determination unit 102 narrows down a plurality of bounding boxes output from the machine learning model 101 to a bounding box that satisfies the first condition that the certainty factor c is higher than the first certainty factor threshold value T_{C1} and the mean value I_{μ} of the luminance values of the input pixels in the bounding box is higher than the first luminance threshold value T_{I1}.

The determination unit 102 performs false positive determination on the affected part candidate narrowed down by first narrowing down under the first condition in this manner.

Note that the first certainty factor threshold value T_{C1} and the first luminance threshold value T_{I1} may be preset to values defined based on experience or experiment, and alterations can be appropriately made and carried out.

The determination unit 102 classifies the plurality of bounding boxes output from the machine learning model 101 into a plurality of groups (size groups) according to the size of each bounding box. For example, the determination unit 102 classifies the bounding box according to the length of one side constituting the bounding box. In the present embodiment, the length of one side of the bounding box is classified into three sizes of a size shorter than 5 mm, a size of 5 mm or longer but shorter than 10 mm, and a size of 10 mm or longer.

A bounding box having a length of the one side shorter than 5 mm may be referred to as an S-sized bounding box. In addition, a bounding box having a length of the one side equal to or longer than 5 mm but shorter than 10 mm may be referred to as an M-sized bounding box, and a bounding box having a length of the one side equal to or longer than 10 mm may be referred to as an L-sized bounding box.

The determination unit 102 sets second certainty factor threshold values T_{C2,SIZE} and second luminance threshold values T_{I2,SIZE} according to the size of the bounding boxes. Hereinafter, the certainty factor threshold value and the luminance threshold value for the S-sized bounding box are represented by reference signs T_{C2,S} and T_{I2,S}, respectively. Similarly, the certainty factor threshold value and the luminance threshold value for the M-sized bounding box are represented by reference signs T_{C2,M} and T_{I2,M}, respectively, and the certainty factor threshold value and the luminance threshold value for the L-sized bounding box are represented by reference signs T_{C2,L} and T_{I2,L}, respectively. The second certainty factor threshold value T_{C2,SIZE} may be simply referred to as a certainty factor threshold value T_{C2,SIZE}. In addition, the second luminance threshold value T_{I2,SIZE} may be simply referred to as a luminance threshold value T_{I2,SIZE}. The second certainty factor threshold value T_{C2,SIZE} corresponds to a second threshold value. In addition, the second luminance threshold value T_{I2,SIZE} corresponds to a fourth threshold value.

In this manner, the determination unit 102 subdivides the threshold values (the certainty factor threshold value and the luminance threshold value) used for false positive determination, according to an index (such as the size of the bounding box) other than the indices (the certainty factor and the luminance) used in the first narrowing down.

For the bounding box of each size, in a case where the certainty factor c is higher than the certainty factor threshold value T_{C2,SIZE} and the mean value I_{μ} of the luminance values of the input pixels in a bounding box is higher than the luminance threshold value T_{I2,SIZE}, the determination unit 102 estimates an affected part candidate included in the bounding box as an aneurysm (true positive).

On the other hand, for the bounding box of each size, in a case where the condition that the certainty factor c is higher than the certainty factor threshold value T_{C2,SIZE} and the mean value I_{μ} of the luminance values of the input pixels in a bounding box is higher than the luminance threshold value T_{I2,SIZE} is not satisfied, the determination unit 102 estimates an affected part candidate included in the bounding box as a false positive.

Note that the certainty factor threshold value T_{C2,SIZE} and the luminance threshold value T_{I2,SIZE} may be preset to values defined based on experience or experiment, and alterations can be appropriately made and carried out.

In the false positive determination, the determination unit 102 determines a false positive of each bounding box (aneurysm candidate) subdivided (classified) according to the size of the bounding box, by comparing the certainty factor c and the mean value I_{μ} of the luminance values of the input pixels in the bounding box with the second threshold values.

The result of the false positive determination by the determination unit 102 may be presented (output) to a user such as a medical doctor, for example. In addition, the result of the false positive determination by the determination unit 102 may be stored in a storage area (not illustrated) of the memory 12, the storage device 13, or the like.

### (B) Operation

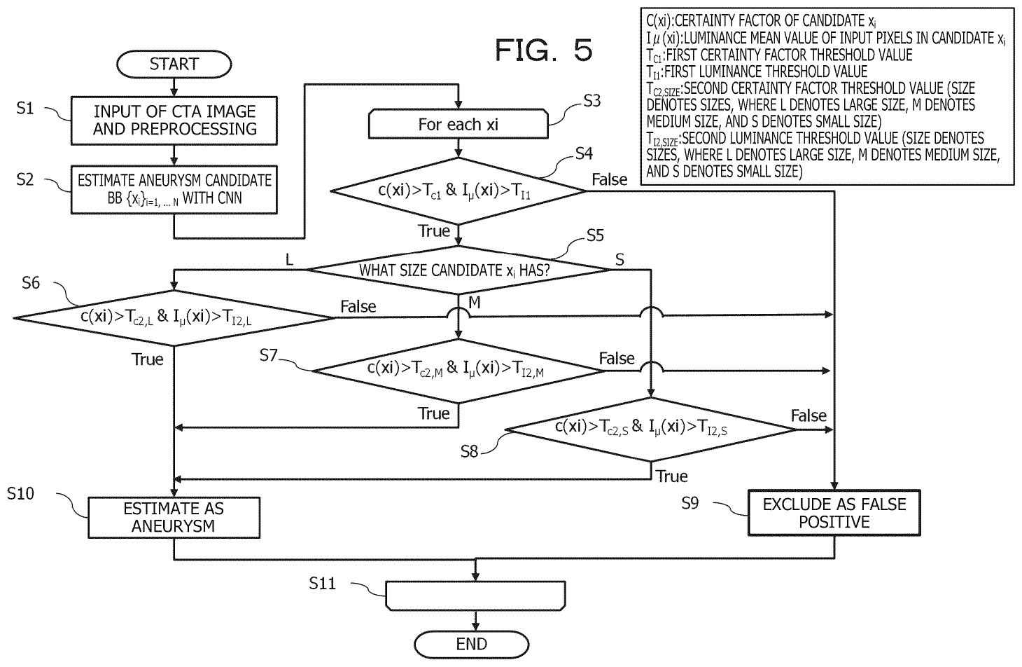

A process in the medical image processing system 1 as an example of the embodiment configured as described above will be described with reference to the flowchart (steps S1 to S11) illustrated in FIG. 5.

In step S1, a CTA medical image is input to the preprocessing unit (not illustrated), and preprocessing such as rescaling is performed. The medical image that has undergone the preprocessing is input to the machine learning model 101.

In step S2, when the medical image is input to the machine learning model 101, the machine learning model 101 estimates an aneurysm candidate in the medical image with a CNN. Any aneurysm candidate among a plurality of (N) aneurysm candidates BB is represented by a reference sign xᵢ. This can be represented as {xᵢ}_{i=1, . . . . N}.

In step S3, a loop process of repeatedly carrying out the control up to step S11 for all the aneurysm candidates BB is started.

In step S4, the determination unit 102 performs the first narrowing down on the aneurysm candidate xᵢ. That is, the determination unit 102 confirms whether the first condition (c(xᵢ)>T_{C1} & I_{μ}(xᵢ)>T_{I1}) that a certainty factor c(xᵢ) of the aneurysm candidate xᵢ is higher than the first certainty factor threshold value T_{C1} and a mean value I_{μ}(xᵢ) of the luminance values of the input pixels in the bounding box is higher than the first luminance threshold value T_{I1} is satisfied.

As a result of this confirmation, in a case where the aneurysm candidate xᵢ does not satisfy the first condition (c(xᵢ)>T_{C1} & I_{μ}(xᵢ)>T_{I1}) (see the False route in step S4), the process proceeds to step S9.

In addition, as a result of the confirmation in step S4, in a case where the aneurysm candidate xᵢ satisfies the first condition (c(xᵢ)>T_{C1} & I_{μ}(xᵢ)>T_{I1}) (see the True route in step S4), the process proceeds to step S5, and the determination unit 102 starts false positive determination for each size group.

In step S5, the determination unit 102 confirms the size of the aneurysm candidate xᵢ. That is, the determination unit 102 confirms the size of the bounding box of the aneurysm candidate xᵢ.

As a result of the confirmation, in a case where the size of the aneurysm candidate xᵢ is the L size (see the “L” route in step S5), the process proceeds to step S6. In addition, in a case where the size of the aneurysm candidate xᵢ is the M size (see the “M” route in step S5), the process proceeds to step S7. Furthermore, in a case where the size of the aneurysm candidate xᵢ is the S size (see the “S” route in step S5), the process proceeds to step S8.

In step S6, the determination unit 102 confirms whether the aneurysm candidate xᵢ satisfies a second condition for the L size (c(xᵢ)>T_{C2,L} & I_{μ}(xᵢ)>T_{I2,L}) that the certainty factor c(xᵢ) is higher than the certainty factor threshold value T_{C2,L} and the mean value I_{μ}(xᵢ) of the luminance values of the input pixels in the bounding box is higher than the luminance threshold value T_{I2,L}.

As a result of this confirmation, in a case where the aneurysm candidate xᵢ does not satisfy the second condition for the L size (see the False route in step S6), the process proceeds to step S9.

In addition, as a result of the confirmation in step S6, in a case where the aneurysm candidate xᵢ satisfies the second condition for the L size (c(xᵢ)>T_{C2,L} & I_{μ}(xᵢ)>T_{I2,L}) (see the True route in step S6), the process proceeds to step S10.

In step S7, the determination unit 102 confirms whether the aneurysm candidate xᵢ satisfies the second condition for the M size (c(xᵢ)>T_{C2,M} & I_{μ}(xᵢ)>T_{I2,M}) that the certainty factor c(xᵢ) is higher than the certainty factor threshold value T_{C2,M} and the mean value I_{μ}(xᵢ) of the luminance values of the input pixels in the bounding box is higher than the luminance threshold value T_{I2,M}.

As a result of this confirmation, in a case where the aneurysm candidate xᵢ does not satisfy the second condition for the M size (see the False route in step S7), the process proceeds to step S9.

In addition, as a result of the confirmation in step S7, in a case where the aneurysm candidate xᵢ satisfies the second condition for the M size (see the True route in step S7), the process proceeds to step S10.

In step S8, the determination unit 102 confirms whether the aneurysm candidate xᵢ satisfies the second condition for the S size (c(xᵢ)>T_{C2,S} & I_{μ}(xᵢ)>T_{I2,s}) that the certainty factor c(xᵢ) is higher than the certainty factor threshold value T_{C2,S} and the mean value I_{μ}(xᵢ) of the luminance values of the input pixels in the bounding box is higher than the luminance threshold value T_{I2,S}.

As a result of this confirmation, in a case where the aneurysm candidate xᵢ does not satisfy the second condition for the S size (see the False route in step S8), the process proceeds to step S9.

In addition, as a result of the confirmation in step S8, in a case where the aneurysm candidate xᵢ satisfies the second condition for the S size (see the True route in step S8), the process proceeds to step S10.

In step S10, the determination unit 102 estimates the aneurysm candidate xᵢ as an aneurysm (true positive). Thereafter, the process proceeds to step S11.

On the other hand, in step S9, the determination unit 102 determines that the aneurysm candidate xᵢ is a false positive and excludes the aneurysm candidate xᵢ. Thereafter, the process proceeds to step S11.

In step S11, a loop end process relevant to step S3 is carried out. Here, when the process for all the aneurysm candidates xᵢ is completed, the present flow is terminated.

### (C) Effects

As described above, according to the medical image processing system 1 as an example of the embodiment, the determination unit 102 determines whether an affected part candidate output from the machine learning model 101 satisfies the first condition that the certainty factor c is higher than the first certainty factor threshold value T_{C1} and the mean value I_{μ} of the luminance values of the input pixels in the bounding box is higher than the first luminance threshold value T_{I1}.

As a result of the determination, the determination unit 102 excludes an affected part candidate included in a bounding box not satisfying this first condition by regarding the affected part candidate as a false positive.

This may enable to exclude the false positive affected part candidate in a short time and to shorten the processing time. In addition, in the false positive determination for each size group of the bounding box to be performed subsequently, the number of affected part candidates to be determined may be reduced, and the load produced in the false positive determination for the affected part candidates may be lessened.

In addition, the determination unit 102 classifies the affected part candidates satisfying the first condition into a plurality of size groups according to the size of its bounding boxes and makes a determination as to whether to be a false positive, using the certainty factor threshold values and the luminance threshold values set for each of these size groups. That is, the determination unit 102 makes a determination as to whether to be a false positive, using the certainty factor threshold values and the luminance threshold values subdivided according to the size of the bounding box.

As described above, by setting the certainty factor threshold values and the luminance threshold values for each size group of the bounding box, an optimal certainty factor threshold value and luminance threshold value may be set in line with the size of the bounding box, and the false positive determination for the affected part candidate may be achieved with high accuracy.

In addition, the determination unit 102 performs the threshold value process on the whole according to the first condition and then subdivides the affected part candidates into different size groups to perform the threshold value process, whereby threshold value setting for each detailed condition may be enabled while the space, time, and computational amount are saved. Furthermore, this may enable to reduce false positives and misses and allow medical doctors to make a definite diagnosis on affected part candidates in a shorter time.

In addition, in the present medical image processing system 1, since a feature or the like indicating position coordinates does not have to be used, special positional alignment is also not involved.

### (D) Others

The disclosed technique is not limited to the embodiments described above, and various modifications can be made and carried out without departing from the spirit of the present embodiments. Each configuration and each process of the present embodiments can be selected or omitted as desired or may be appropriately combined.

For example, in the above-described embodiments, an example in which the medical image is a three-dimensional CTA medical image of the head of the patient and a lesion of a cerebrovascular disease including a cerebral aneurysm is detected is illustrated, but this is not restrictive. For example, the present embodiments may be applied to medical images for other diagnosis approaches such as MRA and additionally, may be applied to two-dimensional medical images. Furthermore, the present embodiments may be applied to detection of a lesion of a cerebrovascular disease other than a cerebral aneurysm or may be applied to detection of a lesion other than a cerebrovascular disease.

In addition, in the above-described embodiments, the determination unit 102 classifies the bounding boxes into three types of size groups of S, M, and L, but this is not restrictive. A plurality of bounding boxes may be classified into two or less types or four or more types of size groups, and alterations can be appropriately made and carried out.

In the above-described embodiments, the determination unit 102 uses the mean value I_{μ} of the luminance values of the input pixels in the bounding box, as the statistical value of the luminance values in the first narrowing down and the false positive determination, but this is not restrictive. The determination unit 102 may use other statistical values such as the maximum value, the minimum value, and the median value of the luminance values of the input pixels in the bounding box, and alterations can be appropriately made and carried out.

In the above-described embodiments, the determination unit 102 classifies the bounding boxes according to the length of one side constituting the bounding box, but this is not restrictive. For example, the bounding boxes may be classified based on a diagonal length or the like of the bounding box, and alterations can be appropriately made and carried out.

In the above-described embodiments, the determination unit 102 subdivides the threshold values (the certainty factor threshold values and the luminance threshold values) used for the false positive determination according to the size of the bounding box, but this is not restrictive. The determination unit 102 may subdivide the threshold values used for the false positive determination, using indices other than the size, certainty factor, and luminance of the bounding box.

In addition, those skilled in the art can carry out or manufacture the present embodiments according to the above disclosure.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a medical image processing program for causing a computer to execute a process comprising:
narrowing down lesion candidate regions detected by a detection model that detects the lesion candidate regions included in a medical image,
by using a first threshold value on certainty factors calculated by the detection model; and
determining false positives of the lesion candidate regions by comparing the certainty factors calculated for the lesion candidate regions with a second threshold value subdivided according to an index other than the certainty factors.

2. The non-transitory computer-readable recording medium according to claim 1, wherein
the narrowing down includes narrowing down that uses a third threshold value on luminance values of pixels in the lesion candidate regions.

3. The non-transitory computer-readable recording medium according to claim 1, wherein
the determining the false positives of the lesion candidate regions includes comparing luminance values of pixels in the lesion candidate regions with a fourth threshold value subdivided according to the index other than the certainty factors.

4. The non-transitory computer-readable recording medium according to claim 1, wherein
the index other than the certainty factors includes a size of the lesion candidate regions.

5. A medical image processing method comprising:
narrowing down lesion candidate regions detected by a detection model that detects the lesion candidate regions included in a medical image,
by using a first threshold value on certainty factors calculated by the detection model; and
determining false positives of the lesion candidate regions by comparing the certainty factors calculated for the lesion candidate regions with a second threshold value subdivided according to an index other than the certainty factors.

6. The medical image processing method according to claim 5, wherein
the narrowing down includes narrowing down that uses a third threshold value on luminance values of pixels in the lesion candidate regions.

7. The medical image processing method according to claim 5, wherein
the determining the false positives of the lesion candidate regions includes comparing luminance values of pixels in the lesion candidate regions with a fourth threshold value subdivided according to the index other than the certainty factors.

8. The medical image processing method according to claim 5, wherein
the index other than the certainty factors includes a size of the lesion candidate regions.

9. An information processing device comprising:
a memory; and
a processor coupled to the memory and configured to:
narrow down lesion candidate regions detected by a detection model that detects the lesion candidate regions included in a medical image, by using a first threshold value on certainty factors calculated by the detection model; and
determine false positives of the lesion candidate regions by comparing the certainty factors calculated for the lesion candidate regions with a second threshold value subdivided according to an index other than the certainty factors.

10. The information processing device according to claim 9, wherein
the processor narrows down by using a third threshold value on luminance values of pixels in the lesion candidate regions.

11. The information processing device according to claim 9, wherein
the processor compares luminance values of pixels in the lesion candidate regions with a fourth threshold value subdivided according to the index other than the certainty factors.

12. The information processing device according to claim 9, wherein
the index other than the certainty factors includes a size of the lesion candidate regions.
